SEQUENCE LISTING (10 sequences)

SEQ ID NO: 1 - DNA, 1134 bp, Achromobacter lyticus
  sig_peptide (1)..(72)
  CDS (73)..(1131)
  mat_peptide (595)..(1131)
atgaaaaaaatctcaaaagctggtctgggactggctctggtctgtgctctggcgacgattggaggcaacgcatctgctcagggacatggattaagcggcgaagatctggtttactcttacgatgaaatgtttgattttgatatcgatgcctacctggcaaaacatgcgccgcatctgcataaacatagcgaagaaatctctcattgggccggatattctggcatttcaccgaaagttcttatcgcattaatggaacaacagtcaggagctgtgagcgccaaaagagcaacaaatcgcccgtttggcaaacttgccagagcagatggatttggcgcccaaacacgcgaagtggcgttagctctgagagaatctctttatgaacgcgatccggatggagccaaaggcccggtcacattagccagagcaaacccgctgcaggcactttttgaacgctcaggagataatgaaccggcagcggctttaagaggagatggcgaatttcaacttgtctacggcagattatttaacgaaccgcgccaggcaaaagccgcaagcgatagatttgcgaaagctggaccggatgttcaaccgttatctccgaatggactgcttcagtttccgtttccgagaggcgcatcttggcatgtgggcggagctcatacaaacacaggatcaggcaattatccgatgtcaagcctggatatgtcaagaggcggaggctggggaagcaatcaaaacggcaattgggtttcagcgagcgcggctggatcttttaaacgccattcttcatgctttgctgaaattgttcatacaggcggctggtcaacaacatactaccatctgatgaacatccagtacaatacaggcgcgaacgttagcatgaatacagccatcgcaaacccggctaatacacaagcgcaggctctgtgcaacggaggccaaagcacaggaccgcatgaacattggtcactgaaacagaacggctcattttaccatctgaacggaacatacctttcaggctatagaatcacagcgacaggcagctcttatgatacaaattgtagccgcttttatttgacaaaaaatggacagaactactgctatggttattatgtgaatcctggaccgaactaa

SEQ ID NO: 2 - protein, 353 aa, Achromobacter lyticus
SAQGHGLSGEDLVYSYDEMFDFDIDAYLAKHAPHLHKHSEEISHWAGYSGISPKVLIALMEQQSGAVSAKRATNRPFGKLARADGFGAQTREVALALRESLYERDPDGAKGPVTLARANPLQALFERSGDNEPAAALRGDGEFQLVYGRLFNEPRQAKAASDRFAKAGPDVQPLSPNGLLQFPFPRGASWHVGGAHTNTGSGNYPMSSLDMSRGGGWGSNQNGNWVSASAAGSFKRHSSCFAEIVHTGGWSTTYYHLMNIQYNTGANVSMNTAIANPANTQAQALCNGGQSTGPHEHWSLKQNGSFYHLNGTYLSGYRITATGSSYDTNCSRFYLTKNGQNYCYGYYVNPGPN

SEQ ID NO: 3 - DNA, 1257 bp, Pseudomonas aeruginosa
  sig_peptide (1)..(93)
  CDS (94)..(1254)
  mat_peptide (709)..(1254)
atgcaacataaaagaagccgtgcgatggcgagcccgagaagcccgttcctgtttgtgctgctggccctggcggtgggtggtactgccaacgcgcatgatgatggcctgccggcatttcgttattcagccgaactgctgggtcaactgcagctgccgtctgtggcactgccgctgaatgatgacctgtttctgtatggccgtgatgcggaagcatttgatctggaagcgtatctggcactgaatgcaccggcactgcgtgataaaagcgaatatctggaacattggtcaggctattattctattaatccgaaagttctgctgacactgatggtcatgcaaagcggtccgctgggtgcaccggatgaacgtgcactggcagcaccgctgggccgtctgtcagccaaacgcggttttgatgcgcaggtgcgcgatgttctgcagcagctgtctcgccgttattatggctttgaagaatatcaactgcgccaggcagcagcacgtaaagcagttggcgaagatggtctgaatgcagcatctgcagcgctgctgggcctgctgcgtgaaggtgcaaaagtcagcgcagtgcagggcggtaatccgctgggtgcatatgcccagacctttcagcgcctgtttggtacaccggcggcagaactgctgcagccgtcaaatcgtgttgcacgtcaactgcaggcaaaagcggcactggcaccgccgagcaacctgatgcagctgccgtggcgtcagggctattcatggcagccgaatggtgcacatagcaacacgggctcaggttatccgtatagctcatttgatgccagctatgattggccgcgttggggctctgcaacctatagcgtggttgcagcccatgcgggtacagtccgcgtgctgtctcgttgccaagttcgtgtcacacatccgtctggttgggcaaccaattattatcatatggatcagattcaggtgagcaacggtcagcaggtttcagcagatacgaaactgggcgtttatgcaggtaatatcaacacagccctgtgcgaaggcggttctagcacgggcccgcatctgcatttttctctgctgtataatggtgcgtttgtctcactgcagggcgcatcttttggtccgtatcgcatcaacgtgggcaccagcaattatgataacgattgtcgccgttattacttctacaatcagtctgctggaacaacccactgtgcctttagaccgctgtataatccgggactggctctgtaa

SEQ ID NO: 4 - protein, 387 aa, Pseudomonas aeruginosa
HDDGLPAFRYSAELLGQLQLPSVALPLNDDLFLYGRDAEAFDLEAYLALNAPALRDKSEYLEHWSGYYSINPKVLLTLMVMQSGPLGAPDERALAAPLGRLSAKRGFDAQVRDVLQQLSRRYYGFEEYQLRQAAARKAVGEDGLNAASAALLGLLREGAKVSAVQGGNPLGAYAQTFQRLFGTPAAELLQPSNRVARQLQAKAALAPPSNLMQLPWRQGYSWQPNGAHSNTGSGYPYSSFDASYDWPRWGSATYSVVAAHAGTVRVLSRCQVRVTHPSGWATNYYHMDQIQVSNGQQVSADTKLGVYAGNINTALCEGGSSTGPHLHFSLLYNGAFVSLQGASFGPYRINVGTSNYDNDCRRYYFYNQSAGTTHCAFRPLYNPGLAL

SEQ ID NO: 5 - DNA, 1164 bp, Aeromonas hydrophila
  sig_peptide (1)..(60)
  CDS (61)..(1161)
  mat_peptide (625)..(1161)
atgtctcgtccgatcccgtccctgctgatgctggctctgctgccggctgctggttgggctggcgatattcacgctccgctggctccgtatcattttacggcgcagcaactggcagcatctcaaaccccggcactgccgctggatgaagcacattttgtttttggcgaagccgcgatggcatttgatctgcatgattttctgctgcagcaggccccgcatctgctgccgaaagaagaagtcattctgcattggagcggtatcacgtcactgaatccgcagctgctgctggccctgatggaagcgagctcacagctgatttcagcaccgtctgaacaggccatggcagccccgtttgcgaaactggtgaatgcacgtggctttgataaccagctggaactgatggcccgccagctgtctgaacgtttttatcaggcacgcgcccagcagaaactgatgcaacgttctgcaccggcactggccccgcaggcggcacatcaggccgcgctggcatcaatgctgtctaccagcatgcagcgtcagctgggcgaacagtggcagaccctgtttggtcaagatgcaatgacaagcccgcgcggcggtgcagcagcaccggcagccccgctggcaggcggtcaatttcagctgccgtggcgtcagggctattcttggaaagcgaatggtgcacattctcatacaggcagcggttatccgtattctagcatcgatgtcagctatgattggccgggttggggcggtgcgacctatacagtgacggcggcaaactcaggtaccgtgacagtgtttagccgttgccaggtccgtgtgacagcaaccaatggctgggcgacaaactattatcatatgagcggcatttcagtgcgttctggtgattatgttgccgcggatacaccgatcggcacgtatgcctcaaatcgcaacgaagcgctgtgcgaaggcggttcatctacgggtccgcatctgcattttagcctgctgtataatggcgtttttcagtcactgcagggtcagcgtctgagctcatatgcagttaatgtcggcgccagcaactatgatgataattgtaaccgcttttggctgtataaccaaagaaacggacaacgctactgtgcttggcaaccgctgtataataacggaatcgactaa

SEQ ID NO: 6 - protein, 367 aa, Aeromonas hydrophila
GDIHAPLAPYHFTAQQLAASQTPALPLDEAHFVFGEAAMAFDLHDFLLQQAPHLLPKEEVILHWSGITSLNPQLLLALMEASSQLISAPSEQAMAAPFAKLVNARGFDNQLELMARQLSERFYQARAQQKLMQRSAPALAPQAAHQAALASMLSTSMQRQLGEQWQTLFGQDAMTSPRGGAAAPAAPLAGGQFQLPWRQGYSWKANGAHSHTGSGYPYSSIDVSYDWPGWGGATYTVTAANSGTVTVFSRCQVRVTATNGWATNYYHMSGISVRSGDYVAADTPIGTYASNRNEALCEGGSSTGPHLHFSLLYNGVFQSLQGQRLSSYAVNVGASNYDDNCNRFWLYNQRNGQRYCAWQPLYNNGID

SEQ ID NO: 7 - DNA, 1164 bp, Lysobacter gummosus
  sig_peptide (1)..(99)
  CDS (100)..(1161)
  mat_peptide (628)..(1161)
atgtctaacaaaacaggatctaaccaggcattttctaaaatgggattagcattattaacatgctgcgttttagcagcaatctctggaggagcaggagcagcggaacgtggtctgagcggccaggacctggtttatagctacgatgaaatgtttgatttcgacattgatgcgtacctggcgaaaaacgcgccgcatctgagccgtcacgcggaaagcatcagccattgggcgggttatagcggcattagcccgaaagtgctgatcgcgctgatggaacagcaaagcggcgcgattacccgtaaacatgcagcagcagatgcagcaaaacgtccgtttggtgcactggcgaaagcgaaagatttcaatggtcagacccgtgaagttgcgcaagcgctgcgtgaagcgctgtacgaaaacgacggtccggatgcaaagggtgcagttaccgtggcacgtgcaaatccgctgcaggcactgtttgaacgtgcgggtgcaagccaagcaagcgcaaaactgagcggtgacggcgaatttcagctggtgtatggtcgtctgttcaacgaaccgcgtcaggcacaggcaccgagcgcacgttttgcaaaagcgggtccggatgttcagccgctgagcccgaatggcctgctgcaatttccgtttccgcgtggtgcgcgttggcatgtgggcggtgcgcacaccaacaccggtagcggcaattacccgatgagcagcctggacatgagcctgggcggtggctggggcagcaaccaaagcaatacctgggttagcgcgagcgcgaacggtagctttaaacgtcatagcagctgcttcgcggaaattgtgcacagcggtggctggagcaccacctattaccatctgatgaacattcgttacaataccggtgcgaacgttggcagcaataccgcgattgcaaatccggcaaatacccgtgcacaggcactgtgcaatggtggcagcagcaccggcccgcatgaacactggagcctgaaactgaacggtagcttttatcatctgaatggtgcgtatctgagcggctaccgtatcaccgcgaccggcagcagctatgataccaactgcagccgtttttacctggcgaaaaacggtcaaaattattgcagcggctggttcaccaatccgggtcactaa

SEQ ID NO: 8 - protein, 354 aa, Lysobacter gummosus
AERGLSGQDLVYSYDEMFDFDIDAYLAKNAPHLSRHAESISHWAGYSGISPKVLIALMEQQSGAITRKHAAADAAKRPFGALAKAKDFNGQTREVAQALREALYENDGPDAKGAVTVARANPLQALFERAGASQASAKLSGDGEFQLVYGRLFNEPRQAQAPSARFAKAGPDVQPLSPNGLLQFPFPRGARWHVGGAHTNTGSGNYPMSSLDMSLGGGWGSNQSNTWVSASANGSFKRHSSCFAEIVHSGGWSTTYYHLMNIRYNTGANVGSNTAIANPANTRAQALCNGGSSTGPHEHWSLKLNGSFYHLNGAYLSGYRITATGSSYDTNCSRFYLAKNGQNYCSGWFTNPGH

SEQ ID NO: 9 - DNA, 1164 bp, Lysobacter antibioticus
  sig_peptide (1)..(77)
  CDS (79)..(1161)
  mat_peptide (628)..(1161)
atgaaagcaatctctaaagcaagattaggattattagcatgctgcatcgcagcagcaatcggaggaacagcaacagcaggcggtcgtgatgcgaatgcagcagcaggtctgagcggtcaggatctggtttatagctacgacgaaatgtttgatttcgacaccgcggcgtatctggcgaaacatgcgccgcacctggttcgtcatagcgaaagcattagccactgggcgggttacagcagcattagcccgaaagtgctgatcgcgctgatggaacagcaaagcggtgttgtgagccgtcaacgtgcaagcgcagatgcaatgcgtcgtccgtttggcaaactgagcgcggcgaaagacttcaatagccagacccgtgaagttgcgaccgcgctgcgtcaggcgctgtatgaacaagaagatgcgagcctggcgccgcaaggtcgtgttccgctggcacgtagcaacccgctgcaggcgctgtatctgcaagcaggtgaaagccaggcaagcgcagcactgcgtggtgacggcgaatttcagcaagtgtacggtcgtctgttcaatgaaccgcgtaaggcagcaccggcaagcgcacgttttgcagataccagcgatgttaatgcactggcaggtccggcgaatggctttctgcagttcccgtatccgcgtggcgcgagctggcatgtgggcggtgcgcacaccaacaccggtagcggcaattacccgatgagcagcctggatatgagccgtggcggtggctggggtagcaaccaaagcggcaattgggttagcgcgagcgcgggtggcagctttaaacgtcatagcagctgcttcgcggaagttgtgcacagcggtggctggagcaccacctattaccatatgatgaacctgcaatatggtaccggtgcgagcgtggcagcaaatagccgtattggtaatccggcaaatacccgtgcacaggcactgtgcaatggtggcgcgagcaccggtccgcatgaacactggagcctgaaatataacggcagccattaccacctgaatggtgtttatctgagcggctaccaaatcaccgcgctgggcagcagctatgacaccaactgcagccgtttttacctgagcaaaaatggtagccgttattgcagcggctacttcaccaatccgtaa

SEQ ID NO: 10 - protein, 361 aa, Lysobacter antibioticus
GGRDANAAAGLSGQDLVYSYDEMFDFDTAAYLAKHAPHLVRHSESISHWAGYSSISPKVLIALMEQQSGVVSRQRASADAMRRPFGKLSAAKDFNSQTREVATALRQALYEQEDASLAPQGRVPLARSNPLQALYLQAGESQASAALRGDGEFQQVYGRLFNEPRKAAPASARFADTSDVNALAGPANGFLQFPYPRGASWHVGGAHTNTGSGNYPMSSLDMSRGGGWGSNQSGNWVSASAGGSFKRHSSCFAEVVHSGGWSTTYYHMMNLQYGTGASVAANSRIGNPANTRAQALCNGGASTGPHEHWSLKYNGSHYHLNGVYLSGYQITALGSSYDTNCSRFYLSKNGSRYCSGYFTNP

(19) United States
(12) Patent Application Publication
(10) Pub. No.: US 2024/0117454 A1
(43) Pub. Date: Apr. 11, 2024

(54) LEATHER IMPROVING AGENT

(71) Applicant: Kao Corporation, Chuo-ku, Tokyo (JP)

(72) Inventor: Takahiro Hioki, Wakayama-shi, Wakayama (JP)

(73) Assignee: Kao Corporation, Chuo-ku, Tokyo (JP)

(21) Appl. No.: 17/768,311

(22) PCT Filed: Oct. 29, 2020
(86) PCT No.: PCT/JP2020/040704

§ 371 Date: Apr. 12, 2022

(30) Foreign Application Priority Data
Oct. 30, 2019 (JP) 2019-197738

(51) Int. Cl. C14C 1/08; C12N 9/64

(52) U.S. Cl. CPC C14C 1/08; C12N 9/6489

(57) ABSTRACT

Provided are a leather improving agent which exhibits the effect of suppressing contraction of hide to enlarge the area; and a method for treating leather using the leather improving agent. The leather improving agent comprises an M23A subfamily protease as an active ingredient.

### FIELD OF THE INVENTION

The present invention relates to a leather improving agent which is used for production of natural leather.

### BACKGROUND OF THE INVENTION

Animal natural leather is widely used in daily life for, for example, footwears such as shoes, clothes and accessories such as bags, handbags, clothing, gloves and belts, furniture such as chairs, interiors and car seats, sporting goods, horse harnesses, drums and handicrafts, and is an important material for household items.

Production of natural leather can be divided into four major steps, i.e. 1) a preparatory operation, 2) a tanning step, 3) a retanning/dyeing/fatliquoring step and 4) a finishing step. In the preparatory operation, blood, dirt, salts, pieces of meat, fat and the like deposited on raw hide are removed, the hide is swollen with lime and a sulfide to loosen collagen fibers and also to degrade and remove hair, and undesired protein is degraded and removed by bating, so that the grain side is smoothed.

The bating, which is also referred to as enzymatic degradation, is an enzyme treatment step which is applied to limed and delimed hide for the purpose of, for example, i) removing hair roots, protein degradation products, fat and the like remaining on the hide, ii) removing substances between fibers of the hide, iii) removing elastin fibers causing the hide to contract, and the like, iv) smoothing the grain side of the hide, and v) mildly unraveling collagen fibers.

Therefore, an enzyme used for bating is required to exhibit the above-described effects. For example, it is known that by degrading elastin in leather by an enzyme having an ability to degrade elastin, contraction of the leather can be suppressed to enlarge the area (Patent Literature 1), but simultaneously, there arises a problem that degradation of collagen, which is a main component of the hide, results in a decrease in strength. As a technique for suppressing contraction of hide to enlarge the area while suppressing collagen degradation, a method has been heretofore proposed in which hide is treated with a mixture of a protease and elastase after chrome tanning (Non Patent Literature 2). In such a method, however, the type of tanning step is limited, and it is necessary to change the production process itself.

On the other hand, the M23 family of proteases is a protease family defined in the MEROPS database as proteases capable of degrading a Gly-Gly bond, has degrading activity on elastin and proteoglycan of bacterial cell walls, and is also known as a bacteriolytic enzyme. Among them, β-lytic protease (BLP) belonging to the M23A subfamily has been reported to have strong bacteriolytic activity on gram-positive bacteria such as Bacillus subtilis (Patent Literature 3). It has been recently found that an M23A subfamily protease can be efficiently produced from a culture by introducing an M23A family protease gene into a Bacillus bacterial host and culturing the bacteria (Patent Literature 4).

However, utilization of an M23A family protease as an enzyme for production of leather has not been heretofore reported.
[Patent Literature 1] WO 2001/035901
[Patent Literature 2] WO 2002/088397
[Patent Literature 3] JP-A-4-108387
[Patent Literature 4] WO 2019/142773

### SUMMARY OF INVENTION

The present invention relates to the following 1) to 4).
1) A method for treating leather, comprising a step of bringing an M23A subfamily protease or an enzyme composition comprising the M23A subfamily protease into contact with a hide.
2) A leather improving agent comprising an M23A subfamily protease as an active ingredient.
3) Use of an M23A subfamily protease for producing a leather improving agent.
4) Use of an M23A subfamily protease for improving leather.

### BRIEF DESCRIPTION OF DRAWINGS

FIG. 1 shows degrading activity of BLP on elastin and collagen (relative value to savinase).

FIG. 2 shows a degrading effect on elastin of bovine hide (without enzyme treatment).

FIG. 3 shows a degrading effect on elastin of bovine hide (savinase treatment). The red line indicates a boundary between an elastin-degraded part and an elastin-remaining part.

FIG. 4 shows a degrading effect on elastin of bovine hide (PPE treatment). The red line indicates a boundary between an elastin-degraded part and an elastin-remaining part.

FIG. 5 shows a degrading effect on elastin of bovine hide (BLP treatment).

FIG. 6 shows a degrading effect on elastin of bovine hide (without enzyme treatment).

FIG. 7 shows a degrading effect on elastin of bovine hide (savinase treatment). The red line indicates a boundary between an elastin-degraded part and an elastin-remaining part.

FIG. 8 shows a degrading effect on elastin of bovine hide (PPE treatment). The red line indicates a boundary between an elastin-degraded part and an elastin-remaining part.

FIG. 9 shows a degrading effect on elastin of bovine hide (LgBLP treatment).

### DESCRIPTION OF EMBODIMENTS

The present invention relates to a provision of a leather improving agent which exhibits the effect of suppressing contraction of hide to enlarge the area, and a method for treating leather using the leather improving agent.

The present inventor found that an M23A subfamily protease typified by BLP can efficiently degrade elastin at a deep part of hide with little degradation of collagen, and is useful as an enzyme which exhibits the effect of suppressing contraction of hide.

The enzyme provided by the present invention, i.e. an M23A subfamily protease, has little collagen degrading activity, and has an excellent ability to efficiently degrade elastin at a deep part of hide. By using such an enzyme in a leather treatment process, contraction of hide can be suppressed to enlarge the area.

As used herein, the “hide” means hide of an animal such as bovine, a pig, a deer, sheep, a horse, a goat, a kangaroo or a crocodile.

As used herein, the “identity of at least 80%” for nucleotide sequences or amino acid sequences is an identity of 80% or more, preferably 85% or more, more preferably 90% or more, even more preferably 95, or more, even more preferably 97% or more, even preferably 98% or more, even more preferably 99% or more.

As used herein, the identity between nucleotide sequences or amino acid sequences can be calculated by the Lipman-Pearson method (Science, 1985, 227: 1435-41). Specifically, the identity can be calculated by performing analysis by using the homology analysis (search homology) program of genetic information processing software Genetryx-Win (ver. 5.1.1; software development) and setting the unit size to compare (ktup) to 2.

As used herein, the “position corresponding to . . . ” on amino acid sequences and nucleotide sequences can be determined by aligning a target sequence and a reference sequence (e.g. an amino acid sequence set forth as SEQ ID NO: 2) so as to give maximum homology to conserved amino acid residues or nucleotides present in the amino acid sequences or nucleotide sequences. The alignment can be carried out using a known algorism, and a procedure thereof is known to a person skilled in the art. For example, the alignment can be performed by using the Clustal W multiple alignment program (Thompson, J. D. et al, 1994, Nucleic Acids Res., 22: 4673-4680) by default setting. Alternatively, it is also possible to use Clustal W2 and Clustal omega which are revised editions of Clustal W. Clustal W, Clustal W2 and Clustal omega can be used on the website of, for example, European Bioinformatics Institute (EBI [www.ebi.ac.uk/index.html]) or DNA Data Bank of Japan (DDBJ [www.ddbj.nig.ac.jp/Welcome-j.html]) operated by National institute of Genetics. The position of an amino acid residue or a nucleotide of the target sequence aligned by the alignment to a position corresponding to a certain position on a reference sequence is considered as a “position corresponding to” such a certain position.

As used herein, the “operable linkage” between a control region and a gene means that a gene and a control region are linked to each other in such a manner that the gene can be expressed under control of the control region. A procedure of making the “operable linkage” between a gene and a control region is well known to a person skilled in the art.

The M23A subfamily protease is a protease which has degrading activity on a glycine-glycine bond in a peptide sequence and is classified into an M23A subfamily that is a subfamily of metalloproteases belonging to the M23 family when classified in accordance with the MEROPS database classification method (Rawlings, Neil D., et al. “MEROPS: the database of proteolytic enzymes, their substrates and inhibitors.” Nucleic acids research 42.D1 (2013): D503-D509).

As proteases belonging to the M23A subfamily, beta-lytic metallopeptidase (BLP), Las A protein (LasA) (also referred to as staphylolysin), Aeromonas hydrophila proteinase (AhP) (also referred to as Mername-AA291 peptidase), BLP homologues derived from Lysobacter gummosus (WP_057941690.1) (hereinafter, referred to as LgBLP), BLP homologues derived from Lysobacter antibioticus) (WP 057970430.1) (hereinafter, referred to as LaBLP) and the like are known at this time.

Therefore, the proteases belonging to the M23A subfamily in the present invention include BLP, LasA, AhP, LgBLP and LaBLP as well as polypeptides equivalent in functions to these proteases. It is preferable to appropriately select and use one or more of these proteases, and it is more preferable to use BLP or polypeptides equivalent in functions to BLP.

BLP (MEROPS ID: M23.001) is a polypeptide consisting of the amino acid sequence from the amino acid at position 1 to the amino acid at position 179 of SEQ ID NO: 2. Las A (MEROP S ID: M23.002) is a polypeptide consisting of the amino acid sequence from the amino acid at position 1 to the amino acid at position 182 of SEQ ID NO: 4. AhP (MEROPS ID: M23.003) is a polypeptide consisting of the amino acid sequence from the amino acid at position 1 to the amino acid at position 179 of SEQ ID NO: 6. LgBLP is a polypeptide consisting of the amino acid sequence from the amino acid at position 1 to the amino acid at position 178 of SEQ ID NO: 8. LaBLP is a polypeptide consisting of the amino acid sequence from the amino acid at position 1 to the amino acid at position 178 of SEQ ID NO: 10.

Examples of the polypeptide equivalent in functions to BLP, Las A and AhP include polypeptides which consist of an amino acid sequence having an identity of at least 80% to any one of the amino acid sequence from the amino acid at position 1 to the amino acid at position 179 of SEQ ID NO: 2, the amino acid sequence from the amino acid at position 1 to the amino acid at position 182 of SEQ ID NO: 4 and the amino acid sequence from the amino acid at position 1 to the amino acid at position 179 of SEQ ID NO: 6 and which have degrading activity on a glycine-glycine bond in a peptide sequence.

Preferred examples of the polypeptide equivalent in functions to BLP include polypeptides which consist of an amino acid sequence having an identity of at least 80% to the amino acid sequence from the amino acid at position 1 to the amino acid at position 179 of SEQ ID NO: 2 and preferably having His at positions corresponding to positions 22, 121 and 123 and Asp at a position corresponding to position 36 on the amino acid sequence from the amino acid at position 1 to the amino acid at position 179 of SEQ ID NO: 2 and which have degrading activity on a glycine-glycine bond in a peptide sequence.

Preferred examples of the polypeptide equivalent in functions to Las A include polypeptides which consist of an amino acid sequence having an identity of at least 80% to the amino acid sequence from the amino acid at position 1 to the amino acid at position 182 of SEQ ID NO: 4 and preferably having His at positions corresponding to positions 23, 120 and 122 and Asp at a position corresponding to position 36 on the amino acid sequence from the amino acid at position 1 to the amino acid at position 182 of SEQ ID NO: 4 and which have degrading activity on a glycine-glycine bond in a peptide sequence.

Preferred examples of the polypeptide equivalent in functions to AhP include polypeptides which consist of an amino acid sequence having an identity of at least 80% to the amino acid sequence from the amino acid at position 1 to the amino acid at position 179 of SEQ ID NO: 6 and preferably having His at positions corresponding to positions 21, 118 and 120 and Asp at a position corresponding to position 34 on the amino acid sequence from the amino acid at position 1 to the amino acid at position 179 of SEQ ID NO: 6 and which have degrading activity on a glycine-glycine bond in a peptide sequence.

Examples of the polypeptide equivalent in functions to LgBLP include polypeptides which consist of an amino acid sequence having an identity of at least 80% to the amino acid sequence from the amino acid at position 1 to the amino acid at position 178 of SEQ ID NO: 8 and which have degrading activity on a glycine-glycine bond in a peptide sequence. Examples of the polypeptide equivalent in functions to LaBLP include polypeptides which consist of an amino acid sequence having an identity of at least 80% to the amino acid sequence from the amino acid at position 1 to the amino acid at position 178 of SEQ ID NO: 10 and which have degrading activity on a glycine-glycine bond in a peptide sequence. Whether degrading activity on a glycine-glycine bond is present or not can be determined by, for example, examining degradability of an oligo glycine peptide, a Fret-GGGGG substrate described in Examples, or the like. However, the method for the determination is not limited thereto.

The M23A subfamily proteases described above can be extracted or prepared from microorganisms producing the proteases, or cultures thereof. For example, BLP can be extracted or prepared from Lysobacter sp. (NBRC 12725 or NBRC 12726), Achromobacter lyticus M497-1, Lysobacter sp. IB-9374, Lysobacter gummosus DSMZ 6980 or the like, or a culture thereof; LAS can be extracted or prepared from Pseudomonas aeruginosa PA01, Pseudomonas aeruginosa ATCC 10145, Pseudomonas aeruginosa FRD1 or the like, or a culture thereof; and AhP can be extracted or prepared from Aeromonas hydrophila subsp. hydrophila ATCC 7966, Aeromonas hydrophila (Chester) Stanier (ATCC 51307) or the like, or a culture thereof. The above-described microorganisms can be purchased from public microorganism culture collections.

A microorganism producing such an M23A subfamily protease may be cultured under appropriate conditions using a culture medium containing consumable carbon sources, nitrogen sources, metal salts, vitamins and the like. From the thus-obtained microorganism or culture solution, an enzyme can be recovered and prepared by a common method, and subjected to freeze-drying, spray drying, crystallization or the like to obtain the enzyme in a desired form. For example, the enzyme can be collected and prepared from the culture using normal methods such as separation of microorganisms by centrifugation or filtration, precipitation of the enzyme in the supernatant or the filtrate by addition of a salt such as ammonia sulfate or by addition of an organic solvent such as ethanol, concentration or de-salting using an ultrafiltration membrane, and purification by any of various kinds of chromatography such as ion-exchange or gel permeation chromatography.

Alternatively, the M23A subfamily protease can be produced by chemical synthesis or a biological method using the amino acid sequences (SEQ ID NOS: 2, 4 and 6) described above. For example, in accordance with the method disclosed in Patent Literature 3, an M23A subfamily protease can be obtained by culturing Bacillus bacteria transformed so as to express a polynucleotide encoding the protein prepared by extracting genomic DNA by a conventional method from a microorganism originally producing a target M23A subfamily protease or extracting RNA to synthesize cDNA by reverse transcription, and then preparing a target enzyme from the culture. Examples of the transformed Bacillus bacteria prepared here include Bacillus bacteria obtained by introducing M23A subfamily protease genes (SEQ ID NOS: 1, 3 and 5) operably linked to control regions into genomes or plasmids of host cells, and Bacillus bacteria containing an expression vector in which a target gene is incorporated at an appropriate position.

Here, the “control region” of a gene is a region which has a function of controlling expression of a gene downstream of the region in cells, and preferably has a function of constitutively expressing or highly expressing the downstream gene. Specifically, the control region can be defined as a region which is present upstream of a coding region in the gene and has a function of controlling transcription of the gene by interaction of RNA polymerases. Preferably, the control region of a gene is a region of about 200 to 600 nucleotides upstream of a coding region in the gene. The control region includes a transcription initiation control region and/or a translation initiation control region of a gene, or a region from the transcription initiation control region up to the translation initiation control region. The transcription initiation control region is a region including a promotor and a transcription initiation point, and the translation initiation control region is a site corresponding to the Shine-Dalgarno (SD) sequence forming a ribosome binding site with an initiation codon (Shine, J., Dalgarno, L., Proc. Natl. Acad. Sci. USA., 1974, 71: 1342-1346).

The expression vector containing an M23A subfamily protease gene can be prepared by incorporating an M23A subfamily protease gene into a vector capable of stably holding the gene, replicating and maintaining the gene in a host microorganism and stably expressing the M23A subfamily protease. Examples of the vector include shuttle vectors such as pHA3040SP64, pHSP64R or pASP64 (JP-B-3492935), pHY300PLK (expression vector capable of transforming both Escherichia coli. and Bacillus subtilis; Jpn JGenet, 1985, 60: 235-243) and pAC3 (Nucleic Acids Res, 1988, 16: 8732); and plasmids usable for transformation of Bacillus bacteria, such as pUB110 (J Bacteriol, 1978, 134: 318-329) and pTA10607 (Plasmid, 1987, 18: 8-15). It is also possible to use plasmids derived from Escherichia coli. (e.g. pET22b(+), pBR322, pBR325, pUC57, pUC118, pUC119, pUC18, pUC19 and pBluescript).

Host Bacillus bacteria can be transformed using a protoplast method, a competent cell method, an electroporation method or the like. The host Bacillus bacterium is preferably Bacillus subtilis or a variant strain thereof. Examples thereof include Bacillus subtilis strains in which extracellular protease production is decreased while sufficiently keeping the M23A maturing ability.

The resulting transformant may be cultured under appropriate conditions using a culture medium containing consumable carbon sources, nitrogen sources, metal salts, vitamins and the like. From the thus-obtained culture, an enzyme can be recovered and prepared by a common method, and subjected to freeze-drying, spray drying, crystallization or the like to obtain the enzyme in a desired form. For example, the enzyme can be collected and prepared from the culture using normal methods such as separation of recombinant microorganisms by centrifugation or filtration, precipitation of the enzyme in the supernatant or the filtrate by addition of a salt such as ammonia sulfate or by addition of an organic solvent such as ethanol, concentration or de-salting using an ultrafiltration membrane, and purification by any of various kinds of chromatography such as ion-exchange or gel permeation chromatography.

Alternatively, the M23A subfamily protease can be prepared from an enzyme composition containing the M23A subfamily protease, or the like. For example, BLP can be prepared from Achromopeptidase. Achromopeptidase is a bacteriolytic enzyme derived from Lysobacter enzymogenes, and contains BLP. Achromopeptidase is commercially available from Wako Pure Chemical Industries, Ltd. etc.

As shown in Examples below, an M23A subfamily protease, e.g. BLP, is equivalent in elastin degrading activity to savinase (S8 family protease) which is a commercially available common bating enzyme and degrades elastin, and it has been shown that BLP can degrade elastin at a deeper part of hide as compared to savinase, and hardly degrades collagen.

Patent Literature 2 indicates that treatment with elastase alone may be effective as a method for degrading elastin while suppressing collagen degradation. However, as shown in Examples, it has been confirmed that even when a purified product of pig pancreas-derived elastase (PPE (Si family protease)) which is one of the most common elastases is used, it is not possible to obtain equivalent elastin selectivity and equivalent degrading activity on elastin at a deep part of leather to those of BLP.

Therefore, the M23A subfamily protease is useful as a leather improving enzyme, preferably a bating enzyme, for suppressing contraction of hide or imparting an area-enlarging effect, and can be a leather improving agent, preferably a bating agent.

In the present invention, the “leather improvement” means that elastin at a deep part of hide is degraded to exhibit a contraction-suppressing effect or an area-enlarging effect on hide. The mode of use of the “leather improving agent” is not limited as long as a contraction-suppressing effect or an area-enlarging effect can be imparted to hide, and the leather improving agent can be used before, after or during any of the steps for leather production, and is preferably used in the bating step.

The bating step means an enzyme treatment step carried out in leather production.

The leather improving agent of the present invention may be an M23A subfamily protease alone, or may be an enzyme composition containing an M23A subfamily protease.

Such an enzyme composition may be a solid composition, for example, in the form of powder, or a liquid composition.

To the enzyme composition containing an M23A subfamily protease, surfactants, chelating agents, water-soluble polymers, water-miscible organic solvents, alkali agents, organic acids or salts thereof, enzymes other than M23A subfamily proteases, enzyme stabilizing agents, antioxidants, solubilizers, pH adjusters, buffering agents, preservatives, perfumes, salts, alcohols, sugars, sawdust, clay and the like can be appropriately added in addition to the M23A subfamily protease.

As the surfactant, any surfactants such as anionic surfactants, nonionic surfactants, cationic surfactants, ampholytic surfactants and gemini surfactants can be used alone, or used in combination of two or more thereof. The content of the surfactants in the enzyme composition is preferably from 0.05 mass % to 20 mass %, more preferably from 0.1 mass % to 10 mass %.

Examples of the nonionic surfactant include polyoxyethylene(polyoxyalkylene)alkyl ethers having a hydrocarbon group having 8 or more and 22 or less carbon atoms, preferably a linear alkyl group having 8 or more and 18 or less carbon atoms, and a polyoxyethylene chain in which ethyleneoxy groups are bonded in an amount of 1 mol or more and 20 mol or less on average, and if necessary, a polyalkyleneoxy chain in which alkyleneoxy groups each selected from the group consisting of a propyleneoxy group and a butyleneoxy group are randomly bonded or block-bonded in an amount of 0 mol or more and 5 mol or less on average to ethyleneoxy groups; polyoxyethylene methyl (or ethyl) ether fatty acid esters obtained by reacting ethylene oxide in an amount of 1 mol or more and 20 mol or less with a methyl- or ethyl-esterified product of a fatty acid having 8 or more and 22 or less carbon atoms; fatty acid alkanolamides in which one fatty acid having 8 or more and 18 or less carbon atoms is amide-bonded to a primary or secondary alkanolamine, where the alkanolamide has, as alkanol groups, one or two alkanol groups each having 2 or 3 carbon atoms, which are bonded to a nitrogen atom and optionally have a polyoxyethylene chain having an average addition molar number of 1 or more and 6 or less, and the alkanolamide optionally has an alkyl group having 1 or more and 3 or less carbon atoms; alkyl (poly)glucosides having a linear or branched hydrocarbon group having 8 or more and 22 or less carbon atoms, preferably a linear alkyl group, where the glucose has an average condensation degree of 1 or more and 3 or less; and glycerol fatty acid ester, pentaerythritol fatty acid esters and sorbitan fatty acid ester which mainly contain a monoester and in which glycerin, pentaerythritol or sorbitan as a polyhydric alcohol is ester-bonded to a fatty acid, and ethylene oxide adducts thereof.

Examples of the anionic surfactant include compounds which have an alkyl group or an alkenyl group having 8 or more and 22 or less carbon atoms and an anionic group and which are any of a linear alkyl benzenesulfonic acid salt, an alkyl or alkenyl sulfuric acid ester salt, a polyoxyethylene(polyoxypropylene)alkyl or alkenyl sulfuric acid ester salt, a polyoxyethylene(polyoxypropylene)alkyl or alkenyl ether carboxylic acid salt, an α-olefin sulfonic acid salt, an internal olefin sulfonic acid salt (including HAS isomers) which is a salt of sulfonated internal olefins having an unsaturated bond at position 2 or higher and preferably position 8 or lower, an α-sulfofatty acid salt, an α-sulfofatty acid methyl ester salt, a fatty acid salt, a phosphoric acid ester salt-based surfactant, an acyl alaninate and an acyl taurate.

Here, examples of the salt include salts with alkali metals, and salts with alkaline earth metals. The salts may be of potassium, sodium, calcium and magnesium, or may be of ammonia or a mono- to trialkanolamine having an alkanol group having 2 or more and 4 or less carbon atoms, preferably monoethanol amine or the like, or a salt may be formed by adding an acid, and neutralized by adding an alkali agent or a strong base-weak acid salt such as sodium carbonate in the system.

Examples of the cationic surfactant include quaternary ammonium salts having 1 or more and 3 or less long-chain alkyl groups or alkenyl groups having 8 or more and 25 or less carbon atoms and optionally interrupted by an ester bond, an ether bond or an amide bond between carbon bonds and short-chain groups each selected from the group consisting of methyl, ethyl and hydroxyethyl as residual groups, with a chlorine ion, a bromine ion, a methyl sulfate ion or an ethyl sulfate ion as a counter ion; tertiary amines having 1 or more and 3 or less long-chain alkyl groups or alkenyl groups optionally interrupted by an ester bond, an ether bond or an amide bond between carbon bonds, and short-chain groups each selected from the group consisting of methyl, ethyl and hydroxyethyl as residual groups, and acid salts thereof; mono-long-chain alkyl or alkenyl trimethyl ammonium salts having an alkyl group or an alkenyl group having 8 or more and 25 or less carbon atoms; di-long-chain alkyl or alkenyl dimethyl ammonium salts; mono-long-chain alkyl or alkenyl pyridinium salts; and mono-long-chain alkyl or alkenyl amide propyl dimethyl amines, or acid salts thereof. Preferred examples include chlorine salts or ethylsulfonic acid salts of alkyl (di- or tri)methyl quaternary ammonium having one or two long-chain alkyl groups having 8 to 22 carbon atoms, chlorine salts or ethylsulfuric acid salts of quaternary ammonium having 1 or more and 3 or less alkanoyl oxyethylene groups having 11 to 25 carbon atoms, and an alkyl group having 1 or 2 carbon atoms, or a hydroxyethyl group; and fatty acid amide propyl dimethyl amines having one long-chain alkyl group having 8 to 22 carbon atoms.

Examples of the ampholytic surfactant include compounds having an alkyl group or an alkenyl group having 8 or more and 22 or less carbon atoms, an anionic group and a cationic group, e.g. ampholytic surfactants of alkyl betaine type, alkyl amide betaine type, imidazoline type, alkyl aminosulfone type, alkyl aminocarboxylic acid type, alkyl amide carboxylic acid type, amide amino acid type or phosphoric acid type, such as alkyl acetic acid betaines, alkanol amide propyl acetic acid betaines, alkyl imidazolines and alkyl alanines. Preferred examples include sulfobetaines or carbobetaines having an alkyl group having 10 to 18 carbon atoms.

Examples of the gemini surfactant, which may be classified as a nonionic surfactant in some cases, include compounds which have a polarity depending on pH and which have an alkyl group or an alkenyl group having 8 or more and 22 or less carbon atoms, and an amine oxide group. Preferred examples include alkylamine oxides having one or two alkyl groups or alkenyl groups having 8 or more and 22 or less carbon atoms, which may be bonded through an amide propylene group to a nitrogen atom forming the amine oxide, and having two alkyl groups having 1 or more and 3 or less carbon atoms, and more preferred examples include alkyl dimethylamine oxides having an alkyl group having 8 or more and 18 or less carbon atoms, and fatty acid amide propyl dimethylamine oxides having a fatty acid residue having 8 or more and 18 or less carbon atoms.

Examples of the chelating agent include amino polyacetic acids such as nitrilotriacetic acid, iminodiacetic acid, ethylenediamineacetic acid, diethylenetriaminepentaacetic acid, glycol ether diaminetetraacetic acid, hydroxyethyliminodiacetic acid, triethylenetetraminehexaacetic acid, djenkolic acid and methylglycinediacetic acid, or salts thereof; organic acids such as diglycolic acid, oxydisuccinic acid, carboxymethyloxysuccinic acid, citric acid, lactic acid, tartaric acid, oxalic acid, malic acid, oxydisuccinic acid, gluconic acid, carboxymethylsuccinic acid and carboxymethyltartaric acid, and salts thereof; aminotri(methylenephosphonic acid), 1-hydroxyethylidene-1,1-diphosphonic acid, ethylenediaminetetra(methylenephosphonic acid) and diethylenetriaminepenta(methylenephosphonic acid), and salts thereof. The salts are as described in the section of anionic surfactants, and may be used as acidic pH adjusters.

The content of the chelating agent in the enzyme composition according to the present invention is preferably from 0.001 mass- to 5 mass %, more preferably from 0.005 mass % to 4 mass %, in terms of an acid type.

Examples of the water-soluble polymer include polymer compounds which having (i) a polyether chain moiety composed of a polymerization unit derived from epoxide having 2 to 5 carbon atoms; and (ii) a polymer chain moiety composed of polymerization units derived from one or more unsaturated carboxylic acids selected from the group consisting of acrylic acid, methacrylic acid and maleic acid, and having a graft structure in which one of (i) and (ii) is a main chain and the other is a branched chain (JP-A-2010-275468 and JP-A-10-060496); and water-soluble polymers having an alkylene terephthalate unit and/or an alkylene isophthalate unit, and an oxyalkylene unit and/or a polyoxyalkylene unit (JP-A-2009-155606).

The content of the water-soluble polymer in the enzyme composition according to the present invention is preferably from 0.01 mass % to 10 mass %, more preferably from 0.05 mass % to 5 mass.

Examples of the water-miscible organic solvent include alkanols, alkylene glycols, glycerin, polyalkylene glycols, (poly)alkylene glycol (mono or di)alkyl ethers, alkyl glyceryl ethers, and aromatic ethers of (poly)alkylene glycol. The alkanol is preferably methanol, ethanol, propanol or the like, the alkylene glycol is ethylene glycol, propylene glycol, butylene glycol, hexylene glycol the like, more preferably ethylene glycol or propylene glycol, even more preferably glycerin, and examples of the polyalkylene glycol is preferably diethylene glycol, triethylene glycol, dipropylene glycol, tripropylene glycol, polyethylene glycol, polypropylene glycol, or polyethylene glycol polypropylene glycol which may be a random or block polymer, more preferably diethylene glycol, dipropylene glycol, polyethylene glycol or polypropylene glycol. The (poly)alkylene glycol (mono or di)alkyl ether is preferably polyoxyethylene monobutyl ether having an average addition molar number of 1 or more and 3 or less, polyoxypropylene monopropyl ether having an average addition molar number of 1 or more and 3 or less, or the like, more preferably diethylene glycol monobutyl ether, and the alkyl glyceryl ether is preferably alkyl (poly)glyceryl ether having an alkyl group having 1 or more and 8 or less carbon atoms, more preferably 2-ethylhexyl glyceryl ether or isoamyl glyceryl ether. The aromatic ether of (poly)alkylene glycol is preferably (poly)oxyethylene monophenyl ether having an average addition molar number of 1 or more and 3 or less, (poly)oxyethylene benzyl ether having an average addition molar number of 1 or more and 3 or less, or the like, more preferably monoethylene glycol monophenyl ether, diethylene glycol monophenyl ether, triethylene glycol ether monophenyl ether, ethylene glycol monobenzyl ether, or diethylene glycol monobenzyl ether.

The content of the water-miscible organic solvent in the enzyme composition according to the present invention is preferably from 0.1 mass % to 40 mass %, more preferably from 0.5 mass % to 35 mass %.

Examples of the alkali agent include inorganic alkali agents such as sodium hydroxide, potassium hydroxide, sodium carbonate and potassium carbonate, and alkanolamines having 1 to 3 C2-C4 alkanols, such as monoethanolamine, diethanolamine, triethanolamine, polyoxyalkyleneamines and dimethylaminopropylamine. Monoethanolamine and triethanolamine are preferable.

The content of the alkali agent in the enzyme composition according to the present invention is preferably from 0 mass to 20 mass %, more preferably from 0 mass % to 10 mass %.

Examples of the organic acid or a salt thereof include the above-described chelating agents, and other organic acids or salts thereof may be used. Examples thereof include polyvalent carboxylic acids such as saturated fatty acids, succinic acid, maleic acid, fumaric acid, and salts thereof; and hydroxycarboxylic acids such as citric acid, malic acid, glycolic acid, p-hydroxybenzoic acid, benzoic acid, and salts thereof.

The content of the organic acid or a salt thereof in the enzyme composition according to the present invention is preferably from 0 mass %, to 5 mass %, more preferably from 0 mass % to 3 mass %.

Examples of enzymes other than M23A subfamily proteases include proteolytic enzymes which are used as common bating enzymes, such as papaya enzyme, subtilisin, and pancreatin which is a crude enzyme extracted from the pancreas of a bovine or a pig.

Examples of the enzyme stabilizing agent include boron compounds, calcium ion sources (calcium ion-supplying compounds), hydroxy compounds and formic acid, examples of the antioxidant include butylhydroxytoluene, distyrenated cresol, sodium sulfite and sodium hydrogen sulfite, and examples of the solubilizers include paratoluenesulfonic acid, cumenesulfonic acid, metaxylenesulfonic acid, and benzoic acid salts (also having an effect as a preservative). Further, the enzyme composition of the present invention may contain paraffins such as octane, decane, dodecane and tridecane, olefins such as decene and dodecene, alkyl halides such as methylene chloride and 1,1,1-trichloroethane, non-water-miscible organic solvents such as terpenes such as D-limonene, dyes, perfumes, antibacterial preservatives, antifoaming agents such as silicone, and the like.

The content of the M23A subfamily protease in the enzyme composition according to the present invention is not particularly limited as long as the protease exhibits activity, and the content is preferably from 0.01 g to 500 g, more preferably from 0.1 g to 200 g, even more preferably from 1 g to 100 g, per kg of the enzyme composition.

In another aspect, the present invention provides a method for treating leather using an M23A subfamily protease. The method includes bringing an M23A subfamily protease or an enzyme composition containing the M23A subfamily protease into contact with hide before, after or during any of the steps for leather production. For example, in one aspect, an M23A subfamily protease or an enzyme composition containing the M23A subfamily protease is brought into contact with limed and delimed hide.

The mode of bringing an M23A subfamily protease into contact with hide may be appropriately selected according to a leather type and region, and the treatment temperature, the treatment time, and the amount of the enzyme used may also be arbitrarily set according to the mode of treatment. For example, a solution containing an M23A subfamily protease or an enzyme composition containing the M23A subfamily protease may be applied to or scattered on the flesh side of hide, and left standing at a temperature of from 15 to 40° C. for a certain amount of time (from 1 hour to 5 hours), or hide may be immersed in such a solution, and left standing at a temperature of from 15 to 40° C. for a certain amount of time (from 1 hour to 5 hours).

Regarding the above-described embodiments, the present invention further discloses the following aspects.
<1> A method for treating leather, comprising a step of bringing an M23A subfamily protease or an enzyme composition comprising the M23A subfamily protease into contact with hide.
<2> The method according to <1>, wherein the M23A subfamily protease is one or more selected from the group consisting of the following a) to e):
a) a polypeptide consisting of the amino acid sequence from the amino acid at position 1 to the amino acid at position 179 of SEQ ID NO: 2, or a polypeptide which consists of an amino acid sequence having an identity of at least 80% to the amino acid sequence from the amino acid at position 1 to the amino acid at position 179 of SEQ ID NO: 2 and which has degrading activity on a glycine-glycine bond in a peptide sequence;
b) a polypeptide consisting of the amino acid sequence from the amino acid at position 1 to the amino acid at position 182 of SEQ ID NO: 4, or a polypeptide which consists of an amino acid sequence having an identity of at least 80% to the amino acid sequence from the amino acid at position 1 to the amino acid at position 182 of SEQ ID NO: 4 and which has degrading activity on a glycine-glycine bond in a peptide sequence;
c) a polypeptide consisting of the amino acid sequence from the amino acid at position 1 to the amino acid at position 179 of SEQ ID NO: 6, or a polypeptide which consists of an amino acid sequence having an identity of at least 80% to the amino acid sequence from the amino acid at position 1 to the amino acid at position 179 of SEQ ID NO: 6 and which has degrading activity on a glycine-glycine bond in a peptide sequence;
d) a polypeptide consisting of the amino acid sequence from the amino acid at position 1 to 178 of SEQ ID NO: 8, or a polypeptide which consists of an amino acid sequence having an identity of at least 80% to the amino acid sequence from the amino acid at position 1 to the amino acid at position 178 of SEQ ID NO: 8 and which has degrading activity on a glycine-glycine bond in a peptide sequence; and
e) a polypeptide consisting of the amino acid sequence from the amino acid at position 1 to the amino acid at position 178 of SEQ ID NO: 10, or a polypeptide which consists of an amino acid sequence having an identity of at least 80% to the amino acid sequence from the amino acid at position 1 to the amino acid at position 178 of SEQ ID NO: 10 and which has degrading activity on a glycine-glycine bond in a peptide sequence.
<3> The method according to <2>, wherein the M23A subfamily protease is one or more selected from the group consisting of the polypeptides a), c), d) and e).
<4> A leather improving agent comprising an M23A subfamily protease as an active ingredient.
<5> The leather improving agent according to <4>, wherein the leather improving agent is a bating agent.
<6> The leather improving agent according to <4> or
<5>, wherein the M23A subfamily protease is one or more selected from the group consisting of the following a) to e):
a) a polypeptide consisting of the amino acid sequence from the amino acid at position 1 to the amino acid at position 179 of SEQ ID NO: 2, or a polypeptide which consists of an amino acid sequence having an identity of at least 80% to the amino acid sequence from the amino acid at position 1 to the amino acid at position 179 of SEQ ID NO: 2 and which has degrading activity on a glycine-glycine bond in a peptide sequence;
b) a polypeptide consisting of the amino acid sequence from the amino acid at position 1 to 182 of SEQ ID NO: 4, or a polypeptide which consists of an amino acid sequence having an identity of at least 80, to the amino acid sequence from the amino acid at position 1 to the amino acid at position 182 of SEQ ID NO: 4 and which has degrading activity on a glycine-glycine bond in a peptide sequence;
c) a polypeptide consisting of the amino acid sequence from the amino acid at position 1 to 179 of SEQ ID NO: 6, or a polypeptide which consists of an amino acid sequence having an identity of at least 80% to the amino acid sequence from the amino acid at position 1 to the amino acid at position 179 of SEQ ID NO: 6 and which has degrading activity on a glycine-glycine bond in a peptide sequence;
d) a polypeptide consisting of the amino acid sequence from the amino acid at position 1 to the amino acid at position 178 of SEQ ID NO: 8, or a polypeptide which consists of an amino acid sequence having an identity of at least 80% to the amino acid sequence from the amino acid at position 1 to the amino acid at position 178 of SEQ ID NO: 8 and which has degrading activity on a glycine-glycine bond in a peptide sequence; and
e) a polypeptide consisting of the amino acid sequence from the amino acid at position 1 to the amino acid at position 178 of SEQ ID NO: 10, or a polypeptide which consists of an amino acid sequence having an identity of at least 80% to the amino acid sequence from the amino acid at position 1 to the amino acid at position 178 of SEQ ID NO: 10 and which has degrading activity on a glycine-glycine bond in a peptide sequence.
<7> The leather improving agent according to <:6>, wherein the M23A subfamily protease is one or more selected from the group consisting of the polypeptides a), c), d) and e).
<8> Use of an M23A subfamily protease for producing a leather improving agent.
<9> The use according to <8>, wherein the leather improving agent is a bating agent.
<10> Use of an M23A subfamily protease for improving leather.
<11> The use according to any one of <8> and <9>, wherein the M23A subfamily protease is one or more selected from the group consisting of the following a) to e):
a) a polypeptide consisting of the amino acid sequence from the amino acid at position 1 to the amino acid at position 179 of SEQ ID NO: 2, or a polypeptide which consists of an amino acid sequence having an identity of at least 80% to the amino acid sequence from the amino acid at position 1 to the amino acid at position 179 of SEQ ID NO: 2 and which has degrading activity on a glycine-glycine bond in a peptide sequence;
b) a polypeptide consisting of the amino acid sequence from the amino acid at position 1 to the amino acid at position 182 of SEQ ID NO: 4, or a polypeptide which consists of an amino acid sequence having an identity of at least 80% to the amino acid sequence from the amino acid at position 1 to the amino acid at position 182 of SEQ ID NO: 4 and which has degrading activity on a glycine-glycine bond in a peptide sequence;
c) a polypeptide consisting of the amino acid sequence from the amino acid at position 1 to the amino acid at position 179 of SEQ ID NO: 6, or a polypeptide which consists of an amino acid sequence having an identity of at least 80% to the amino acid sequence from the amino acid at position 1 to the amino acid at position 179 of SEQ ID NO: 6 and which has degrading activity on a glycine-glycine bond in a peptide sequence;
d) a polypeptide consisting of the amino acid sequence from the amino acid at position 1 to the amino acid at position 178 of SEQ ID NO: 8, or a polypeptide which consists of an amino acid sequence having an identity of at least 80% to the amino acid sequence from the amino acid at position 1 to the amino acid at position 178 of SEQ ID NO: 8 and which has degrading activity on a glycine-glycine bond in a peptide sequence; and
e) a polypeptide consisting of the amino acid sequence from the amino acid at position 1 to the amino acid at position 178 of SEQ ID NO: 10, or a polypeptide which consists of an amino acid sequence having an identity of at least 80% to the amino acid sequence from the amino acid at position 1 to the amino acid at position 178 of SEQ ID NO: 10 and which has degrading activity on a glycine-glycine bond in a peptide sequence.
<12> The use according to <11>, wherein the M23A subfamily protease is one or more selected from the group consisting of the polypeptides a), c), d) and e).

### EXAMPLES

### Example 1: Preparation of BLP and LgBLP

### (1-1) Preparation of Transformed Strain

Bacillus subtilis 168 strain (Bacillus subtilis Marburg No. 168 strain: Nature, 390, 1997, p. 249) was used as a host. The plasmids pHY-BLP2 and pHY-LgBLP disclosed in Patent Literature 4, Example 1 were each introduced by the following method. Bacillus subtilis 168 strain was inoculated in 1 mL of LB culture medium, and subjected to shake culturing overnight at 200 rpm at 30° C. 10 μL of the culture solution was inoculated in 1 mL of fresh LB culture medium, and cultured at 200 rpm at 37° C. for 3 hours. This culture solution was centrifuged, and pellets were collected. To the pellets was added 500 μL of SMMP (0.5 M sucrose, 20 mM disodium maleate, 20 mM magnesium chloride hexahydrate salt and 35% (w/v) antibiotic medium 3 (Difco)) containing lysozyme (SIGMA) at 4 mg/mL, and the mixture was incubated at 37° C. for 1 hour. Next, pellets were collected by centrifugation, and suspended in 400 μL of SMMP. 33 μL of the suspension liquid and 20 ng of each plasmid were mixed, 100 μL of 40% PEG was added thereto and stirred, 350 μL of SMMP was further added thereto, and the mixture was then shaken at 30° C. for 1 hour. 200 μL of this liquid was applied to DM3 recycled agar culture medium (0.8% agar (Wako Pure Chemical Industries, Ltd), 0.5% disodium succinate hexahydrate salt, 0.5% casamino acid technical (Difco), 0.5% yeast extract, 0.35% monopotassium phosphate, 0.15% dipotassium phosphate, 0.5% glucose, 0.4% magnesium chloride hexahydrate salt, 0.01% bovine serum albumin (SIGMA), 0.5% carboxymethyl cellulose, 0.005% trypan blue (Merck) and amino acid mixture (tryptophane, lysine and methionine at 10 μL/mL each); % refers to (w/v) %) containing tetracycline (15 μg/mL, SIGMA), and was incubated at 30° C. for 3 days to form a colony.

### (1-2) Culture for Enzyme Production

In 1 mL of LB culture medium to which tetracycline had been added to a final concentration of 15 ppm, the recombinant Bacillus subtilis colony obtained in (1-1) was inoculated, and then cultured overnight at 150 spm at 30° C. On the following day, 400 μL of the culture solution was inoculated in 5 mL of 2×L-maltose culture medium (2% tryptone, 1 yeast extract, 1% NaCl, 7.5% maltose, 7.5 ppm manganese sulfate pentahydrate, 21 μM ZnSO₄, 15 ppm tetracycline; % refers to (w/v) %), and cultured at 150 spm at 30° C. for 2 days, and a culture supernatant containing an enzyme produced from bacterial cells was collected by centrifugation.

### (1-3) Purification of Enzyme from Culture Supernatant

BLP and LgBLP were each purified from the culture supernatant obtained in (1-2). The culture supernatant was subjected to buffer exchange with 10 mM citric acid-Na pH 6 using Amicon Ultra (molecular weight cutoff 10 K) (Merck Millipore). From the solution after buffer exchange, an enzyme was purified using AKTA explorer 10S (GE Healthcare). The solution obtained by the buffer exchange was caused to pass through TOYOPEARL Gigacap CM-650M Column (TOSOH CORPORATION), and adsorbed components were then eluted using an elution buffer (10 mM citric acid-Na pH 6, 200 mM NaCl). Among the eluted fractions, fraction solutions having degrading activity on FRET-GGGGG (Examples 1 to 4) were collected. Using Amicon Ultra (molecular weight cutoff 10 K), the collected fraction solutions were subjected to buffer exchange with a 20 mM Tris-HCL (pH 7.5) solution to obtain enzyme solutions containing BLP and LgBLP.

### (1-4) Measurement of Enzyme Activity

As a substrate, a FRET substrate having pentaglycine between the fluorescence group Nma and the quenching group Lys (Dpn) [hereinafter, FRET-GGGGG] (produced on order in PH Japan Co., Ltd.) was used. Here, Nma refers to 2-(N-methylamino)benzoyl (Nma). Lys (Dpn) refers to a group having 2,4-dinitrophenyl (Dnp) on a side chain of lysine (Lys). To a 96-well black plate, 2 μL of the enzyme solution (appropriately diluted) and 200 μL of 20 mM Tris-HCl (pH 7.5) were added, and 10 μL of a FRET-GGGGG solution (1 mM FRET-GGGGG and 100 mM Tris-HCl (pH 7.5)) was further added to prepare a reaction solution. The fluorescence intensity of the reaction solution was measured with time at a temperature of 30° C., an excitation wavelength of 340 nm and a measurement wavelength of 440 nm using infinite M200 (TECAN). Under the same conditions, the fluorescence intensity of a reaction solution obtained using 20 mM Tris-HCl (pH 7.5) instead of the enzyme solution and equimolar solutions of FRETS-25-STD1 and FRETS-25-STD2 (PEPTIDE INSTITUTE, INC.) instead of FRET-GGGGG was measured to prepare a calibration curve. One unit (U) of activity was an enzyme amount required to change the fluorescence intensity by X/min where X is a fluorescence intensity of a solution containing 1 μmol FRETS-25-STD1 and 1 μmol FRETS-25-STD2. The FRET-GGGGG degrading activity (U/mL) of the enzyme solution was determined.

### (1-5) Measurement of Concentration of Enzyme Solution

DC Protein Assay Kit (Bio-Rad) was used for measurement of the concentration of the enzyme solution. BSA Standard Solution (WAKO) was used as a standard solution for calculation of the protein amount.

### Example 2: Measurement of Degrading Activity on Elastin and Collagen

BLP, LgBLP, savinase (SIGMA, P3111) and pig pancreas-derived purified elastase (hereinafter, PPE) (Worthington Biochemical, ESFF) were used as proteases. Bacillus-derived subtilisin containing savinase is a common bating enzyme (Tanning Chemistry: The Science of Leather).

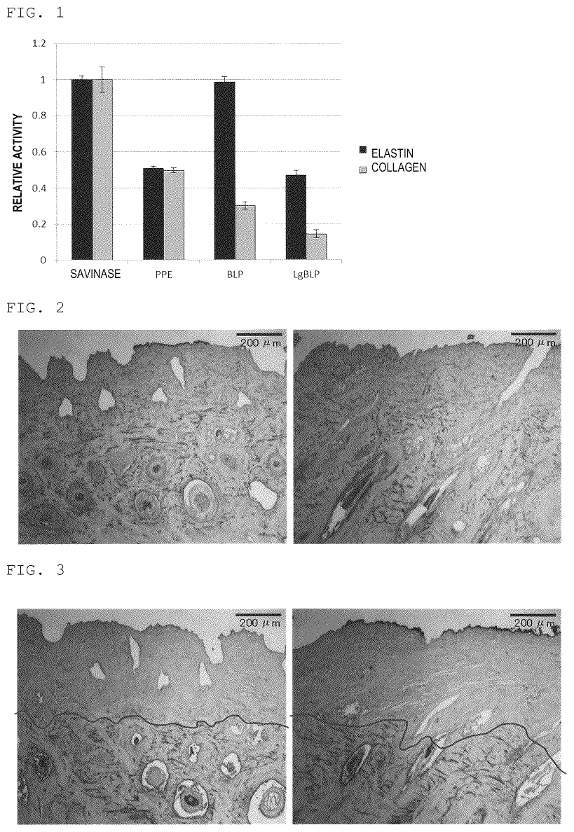

Bovine neck ligament-derived elastin (SIGMA, E1625) and bovine Achilles tendon-derived collagen (SIGMA, C9879) were used as substrates. To 1 mL of 50 mM Tris-HCl pH 7.5 containing 20 mg of the substrate, each enzyme was added to a final concentration of 1 mg/L. The mixture was reacted at 30° C. for 1 hour, and then centrifuged at 15,000 rpm at 4° C. for 5 minutes, and the supernatant was collected. The amount of peptides in the supernatant was determined using TaKaRa BCA Protein Assay Kit (TaKaRa). An enzyme-free sample was defined as a blank, and the blank value was subtracted to calculate the amount of peptides liberated by protease activity. This amount is defined as degrading activity, and is shown as a relative value to savinase in FIG. 1.

BLP was equivalent in elastin degrading activity to savinase, and had significantly lower collagen degrading activity as compared to savinase. LgBLP had lower elastin degrading activity as compared to savinase, and was as high as BLP in selectivity for elastin. PPE had lower elastin degrading activity and collagen degrading activity as compared to savinase. PPE was equivalent to savinase in selectivity for elastin and collagen.

### Example 3: Test on Degradation of Bovine Hide by BLP

### (3-1) Preparation of Bovine Hide for Bating

Lined, dehaired and limed bovine hide was cut to 1.5 cm square and used. Twelve hide pieces were put in a centrifuge tube, immersed in 40 mL of an aqueous ammonium chloride solution containing ammonium chloride at 3% (w/w) of the hide weight, and incubated at room temperature for 60 minutes to perform deliming. The delimed hide pieces were rinsed once with 50 mM Tris-HCl pH 7.5, and used for the following tests.

### (3-2) Protease Treatment of Bovine Hide

Two of the hide pieces prepared in (3-1) were put in each screw tube bottle (27 mm×55 mm) containing 5 mL of 50 mM Tris-HCl pH 7.5. To the screw tube bottle, proteases (savinase, PPE and BLP) were added to a final concentration of 50 mg/L to start a reaction. Incubation was performed at 150 spm at 30° C. for 4 hours, and each hide piece was transferred to 10 mL of 0.1 M sulfuric acid to stop the reaction.

### (3-3) Staining of Tissue

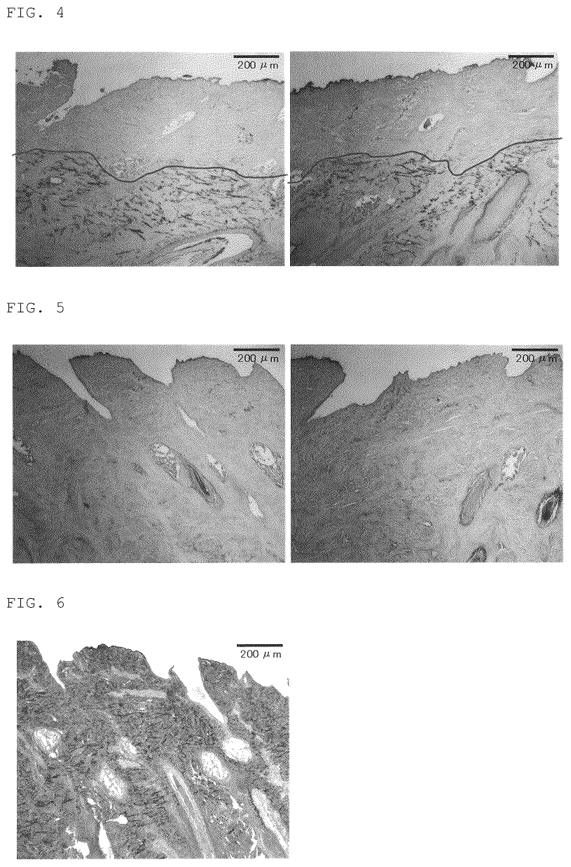

A paraffin block was prepared using the protease-treated hide piece obtained in (3-2). The paraffin block was sliced, and an orcein-stained specimen was then prepared, and observed with a microscope. Elastin is stained brown-black in orcein staining. In a sample treated only with a buffer (FIG. 2), a distribution of elastin was observed on the grain side. In a savinase-treated sample (FIG. 3) and a PPE-treated sample (FIG. 4), it was observed that elastin was degraded only at a surface part of the grain side (a red line is drawn on a boundary between the elastin-degraded part and the elastin-remaining part). In a BLP-treated sample (FIG. 5), elastin even at a deep part was degraded, and remaining of elastin under the visual field in FIG. 5 was not observed. FIGS. 2 to 5 each show the images of stained tissues of two hide pieces each independently subjected to protease treatment (right picture and left picture in each figure).

### Example 4: Test on Degradation of Bovine Hide by LgBLP

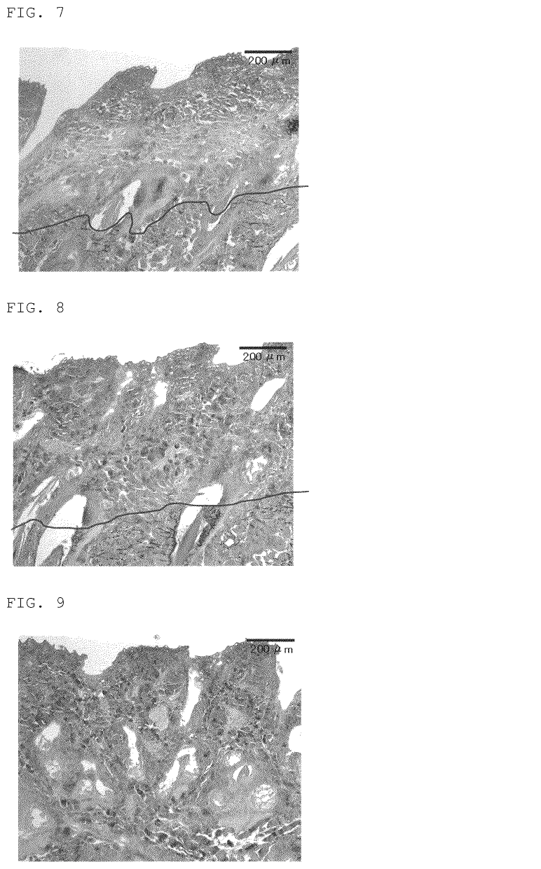

Hide pieces were prepared in the same manner as in (3-1). One of the prepared hide pieces was put in a 12-well microplate to which 2 mL of 50 mM Tris-HCl pH 7.5 was dispensed. Proteases (savinase, PPE and LgBLP) were added to each of the wells to start a reaction. The concentrations of the proteases added were each set to a concentration at which elastin degrading activity equivalent to that of savinase at 50 mg/L was exhibited (calculated from the results of Example 2). Incubation was performed at 150 spm at 30° C. for 4 hours, and each hide piece was transferred to 10 mL of 0.1 M sulfuric acid to stop the reaction. An orcein-stained specimen was prepared in the same manner as in (3-3), and observed with a microscope. In a sample treated only with a buffer (FIG. 6), a distribution of elastin was observed on the grain side. In a savinase-treated sample (FIG. 7) and a PPE-treated sample (FIG. 8), it was observed that elastin was degraded only at a surface part of the grain side (a red line is drawn on a boundary between the elastin-degraded part and the elastin-remaining part). In a LgBLP-treated sample (FIG. 9), elastin even at a deep part was degraded, and remaining of elastin under the visual field in FIG. 9 was not observed.

1. A method for treating leather, comprising a step of bringing an M23A subfamily protease or an enzyme composition comprising the M23A subfamily protease into contact with hide.

2. The method according to claim 1, wherein the M23A subfamily protease is one or more selected from the group consisting of the following a) to e):
a) a polypeptide consisting of the amino acid sequence from the amino acid at position 1 to the amino acid at position 179 of SEQ ID NO: 2, or a polypeptide which consists of an amino acid sequence having an identity of at least 80% to the amino acid sequence from the amino acid at position 1 to the amino acid at position 179 of SEQ ID NO: 2 and which has degrading activity on a glycine-glycine bond in a peptide sequence;
b) a polypeptide consisting of the amino acid sequence from the amino acid at position 1 to the amino acid at position 182 of SEQ ID NO: 4, or a polypeptide which consists of an amino acid sequence having an identity of at least 80% to the amino acid sequence from the amino acid at position 1 to the amino acid at position 182 of SEQ ID NO: 4 and which has degrading activity on a glycine-glycine bond in a peptide sequence;
c) a polypeptide consisting of the amino acid sequence from the amino acid at position 1 to the amino acid at position 179 of SEQ ID NO: 6, or a polypeptide which consists of an amino acid sequence having an identity of at least 80% to the amino acid sequence the amino acid at position 1 to the amino acid at position 179 of SEQ ID NO: 6 and which has degrading activity on a glycine-glycine bond in a peptide sequence;
d) a polypeptide consisting of the amino acid sequence the amino acid at position 1 to the amino acid at position 178 of SEQ ID NO: 8, or a polypeptide which consists of an amino acid sequence having an identity of at least 80% to the amino acid sequence the amino acid at position 1 to the amino acid at position 178 of SEQ ID NO: 8 and which has degrading activity on a glycine-glycine bond in a peptide sequence; and
e) a polypeptide consisting of the amino acid sequence from the amino acid at position 1 to the amino acid at position 178 of SEQ ID NO: 10, or a polypeptide which consists of an amino acid sequence having an identity of at least 80% to the amino acid sequence from the amino acid at position 1 to the amino acid at position 178 of SEQ ID NO: 10 and which has degrading activity on a glycine-glycine bond in a peptide sequence.

3. The method according to claim 2, wherein the M23A subfamily protease is one or more selected from the group consisting of the polypeptides a), c), d) and e).

4. A leather improving agent comprising an M23A subfamily protease as an active ingredient.

5. The leather improving agent according to claim 4, wherein the leather improving agent is a bating agent.

6. The leather improving agent according to claim 4, wherein the M23A subfamily protease is one or more selected from the group consisting of the following a) to e):
a) a polypeptide consisting of the amino acid sequence from the amino acid at position 1 to the amino acid at position 179 of SEQ ID NO: 2, or a polypeptide which consists of an amino acid sequence having an identity of at least 80% to the amino acid sequence from the amino acid at position 1 to the amino acid at position 179 of SEQ ID NO: 2 and which has degrading activity on a glycine-glycine bond in a peptide sequence;
b) a polypeptide consisting of the amino acid sequence from the amino acid at position 1 to the amino acid at position 182 of SEQ ID NO: 4, or a polypeptide which consists of an amino acid sequence having an identity of at least 80% to the amino acid sequence from the amino acid at position 1 to the amino acid at position 182 of SEQ ID NO: 4 and which has degrading activity on a glycine-glycine bond in a peptide sequence;
c) a polypeptide consisting of the amino acid sequence from the amino acid at position 1 to the amino acid at position 179 of SEQ ID NO: 6, or a polypeptide which consists of an amino acid sequence having an identity of at least 80% to the amino acid sequence from the amino acid at position 1 to the amino acid at position 179 of SEQ ID NO: 6 and which has degrading activity on a glycine-glycine bond in a peptide sequence;
d) a polypeptide consisting of the amino acid sequence from the amino acid at position 1 to the amino acid at position 178 of SEQ ID NO: 8, or a polypeptide which consists of an amino acid sequence having an identity of at least 80% to the amino acid sequence from the amino acid at position 1 to the amino acid at position 178 of SEQ ID NO: 8 and which has degrading activity on a glycine-glycine bond in a peptide sequence; and
e) a polypeptide consisting of the amino acid sequence from the amino acid at position 1 to the amino acid at position 178 of SEQ ID NO: 10, or a polypeptide which consists of an amino acid sequence having an identity of at least 80% to the amino acid sequence from the amino acid at position 1 to the amino acid at position 178 of SEQ ID NO: 10 and which has degrading activity on a glycine-glycine bond in a peptide sequence.

7. The leather improving agent according to claim 6, wherein the M23A subfamily protease is one or more selected from the group consisting of the polypeptides a), c), d) and e).

8.-10. (canceled)

11. The leather improving agent according to claim 5, wherein the M23A subfamily protease is one or more selected from the group consisting of the following a) to e):
a) a polypeptide consisting of the amino acid sequence from the amino acid at position 1 to the amino acid at position 179 of SEQ ID NO: 2, or a polypeptide which consists of an amino acid sequence having an identity of at least 80% to the amino acid sequence from the amino acid at position 1 to the amino acid at position 179 of SEQ ID NO: 2 and which has degrading activity on a glycine-glycine bond in a peptide sequence;
b) a polypeptide consisting of the amino acid sequence from the amino acid at position 1 to the amino acid at position 182 of SEQ ID NO: 4, or a polypeptide which consists of an amino acid sequence having an identity of at least 80% to the amino acid sequence from the amino acid at position 1 to the amino acid at position 182 of SEQ ID NO: 4 and which has degrading activity on a glycine-glycine bond in a peptide sequence;
c) a polypeptide consisting of the amino acid sequence from the amino acid at position 1 to the amino acid at position 179 of SEQ ID NO: 6, or a polypeptide which consists of an amino acid sequence having an identity of at least 80% to the amino acid sequence from the amino acid at position 1 to the amino acid at position 179 of SEQ ID NO: 6 and which has degrading activity on a glycine-glycine bond in a peptide sequence;
d) a polypeptide consisting of the amino acid sequence from the amino acid at position 1 to the amino acid at position 178 of SEQ ID NO: 8, or a polypeptide which consists of an amino acid sequence having an identity of at least 80% to the amino acid sequence from the amino acid at position 1 to the amino acid at position 178 of SEQ ID NO: 8 and which has degrading activity on a glycine-glycine bond in a peptide sequence; and
e) a polypeptide consisting of the amino acid sequence from the amino acid at position 1 to the amino acid at position 178 of SEQ ID NO: 10, or a polypeptide which consists of an amino acid sequence having an identity of at least 80% to the amino acid sequence from the amino acid at position 1 to the amino acid at position 178 of SEQ ID NO: 10 and which has degrading activity on a glycine-glycine bond in a peptide sequence.
